(19) United States
(12) Patent Application Publication
(10) Pub. No.: US 2018/0072666 A1
(43) Pub. Date: Mar. 15, 2018

(54) ENOYL REDUCTASE INHIBITORS WITH ANTIBACTERIAL ACTIVITY

(71) Applicant: Novalex Therapeutics, Inc., Chicago, IL (US)

(72) Inventors: Shahila Mehboob Christie, Chicago, IL (US); Jinhong Ren, Chicago, IL (US); Michael E. Johnson, Chicago, IL (US)

(21) Appl. No.: 15/701,725

(22) Filed: Sep. 12, 2017

Related U.S. Application Data
(60) Provisional application No. 62/393,963, filed on Sep. 13, 2016.

(51) Int. Cl. C07D 207/08; C07D 401/06; C07D 401/14; C07D 405/14; C07D 417/14; C07D 417/04; C07D 405/04; C07D 401/04; C07D 207/09

(52) U.S. Cl. CPC C07D 207/08; C07D 401/06; C07D 401/14; C07D 405/14; C07D 207/09; C07D 417/04; C07D 405/04; C07D 401/04; C07D 417/14

(57) ABSTRACT

The present disclosure provides FabI inhibitors and methods of treating or preventing a disease or disorder in a subject by administering same.

### RELATED APPLICATIONS

This application claims priority to U.S. Provisional Patent Application Ser. No. 62/393,963 filed Sep. 13, 2016, the contents of which are hereby incorporated by reference in its entirety.

### TECHNICAL FIELD

The present disclosure relates to a series of substituted pyrrolidine analogs, their tautomers, polymorphs, stereoisomers, prodrugs, solvates, pharmaceutically acceptable salts, pharmaceutical compositions containing them and methods of treating conditions and diseases that are mediated by the inhibition of enoyl reductase or FabI. These compounds are useful in the treatment, prevention or suppression of bacterial diseases.

### BACKGROUND

Antibacterial resistance is on the rise globally with the looming threat of the world entering the pre-antibiotic era. The World Health Organization has declared antimicrobial resistance to be one of the three most important threats to human health.

The bacterial fatty acids synthesis pathway is a particularly attractive drug target. This is because not only is it essential in many bacteria but it differs significantly from the human counterpart as the bacterial fatty acid synthesis (FAS II system) is carried out by a series of discrete enzymes whereas in mammals it takes place on a single, multi-enzyme complex (FAS-I system). The FAS I and FAS II enzymes are structurally and mechanistically distinct, which strongly indicates the possibility of selective antimicrobial targeting of the bacterial pathogens. Enoyl-ACP reductase I or FabI catalyzes the final rate-limiting step in the FAS-II elongation cycle. The FabI enzyme is a member of the short-chain alcohol dehydrogenase/reductase (SDR) superfamily and is responsible for the reduction of a key double bond in the enoyl substrate utilizing NADH or NADPH as a cofactor depending on the bacterial species. FabI is an excellent target for obtaining narrow spectrum therapeutics specifically targeting the pathogen of interest with minimal disruption of the human gut microbiome.

FabI is recognized as a novel drug target for the development of antibiotics. See for example U.S. Pat. Nos. 8,710,073; 6,613,553; 6,881,553; 6,531,291.

### SUMMARY

In some embodiments, the present disclosure provides a potent inhibitor of FabI useful in the treatment of the diseases caused by organisms that utilize FabI as their primary enoyl reductase, including, but not limited to one or more of the following organisms: Francisella tularensis, Staphylococcus aureus, Bacillus anthraces, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsiarickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia coli, Enterobacter Klebsiella pneumoniae, and Listeria monocytogenes.

The present disclosure provides compounds of Formulas (I)-(IV), their tautomers, polymorphs, stereoisomers, prodrugs, solvates, pharmaceutically acceptable salts, pharmaceutical compositions containing them and methods of treating conditions and diseases that are mediated by FabI inhibitory activity.

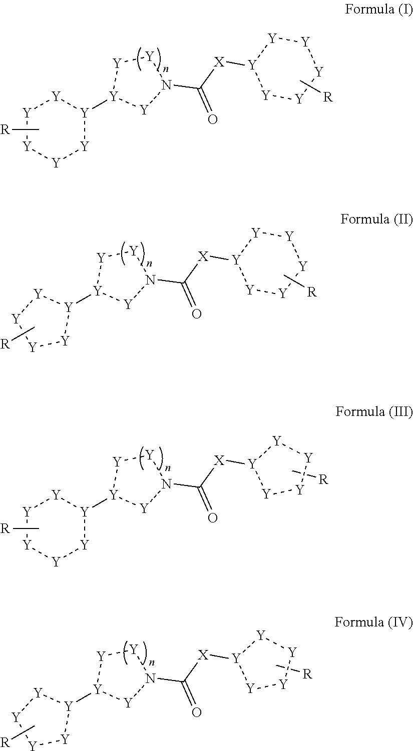

wherein for each of Formulas (I)-(IV):
each ---- independently represents a single bond or a double bond;
X=CH₂, CHR, CR₂, or CH═CH;
each Y is independently CR^{a}R^{b}, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

In some embodiments, the

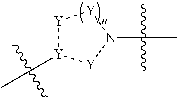

moiety of the compound of Formulas (I)-(IV) is selected from the group consisting of:

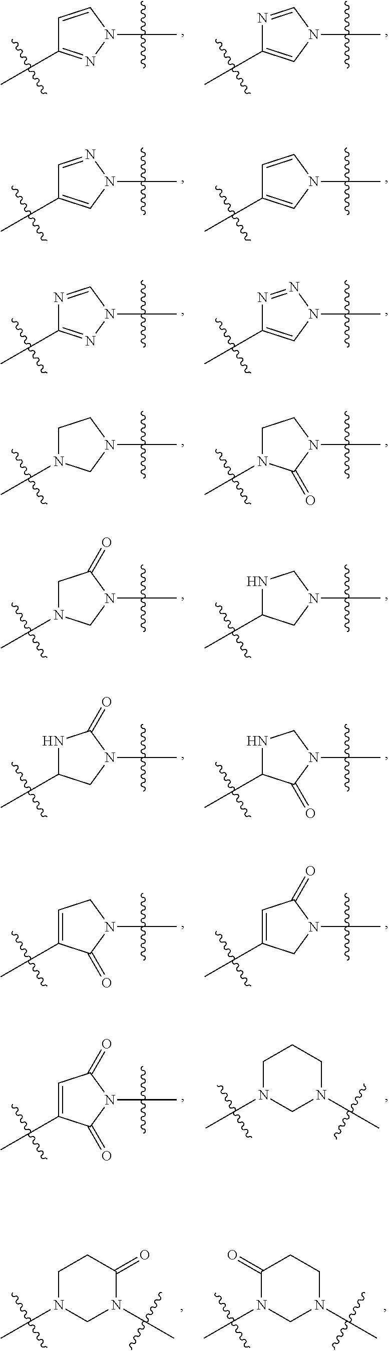

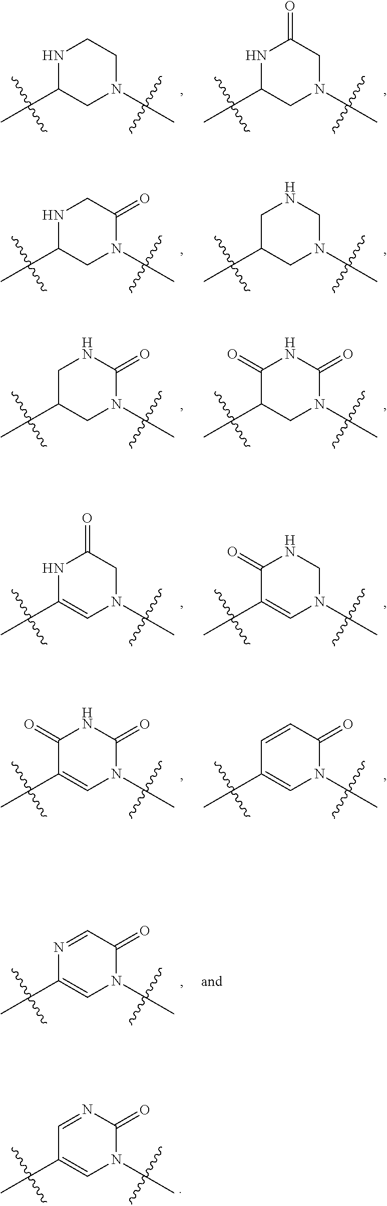

In some embodiments, the present disclosure provides a method of treating or preventing a disease or disorder in a subject, the method comprising administering to the subject an effective amount of at least one compound of Formula (I), Formula (II), Formula (III), and/or Formula (IV), or a pharmaceutical composition comprising at least one compound of Formula (I), Formula (II), Formula (III), and/or Formula (IV).

### DETAILED DESCRIPTION

The present disclosure provides potent inhibitors of FabI. Generally, the FabI inhibitors are di-substituted pyrrolidine analogs or di-substituted piperidine analogs comprising an N-acyl substituent and an aliphatic or aromatic ring at the 3-position.

In some embodiments, the FabI inhibitor is a compound according to Formula (I):

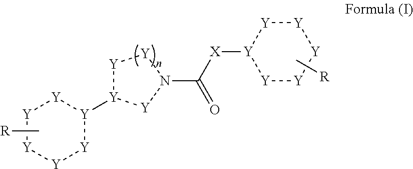

wherein:
each ---- independently represents a single bond or a double bond;
X=CH₂, CHR, CR₂, or CH═CH;
each Y is independently CR^{a}R^{b}, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

In some embodiments, the FabI inhibitor is a compound according to Formula (II):

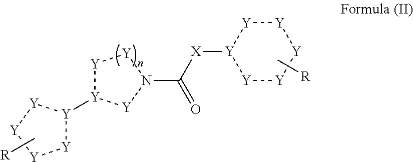

wherein:
each ---- independently represents a single bond or a double bond;
X=CH₂, CHR, CR₂, or CH═CH;
each Y is independently CR^{a}R^{b}, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

In some embodiments, the FabI inhibitor is a compound according to Formula (III):

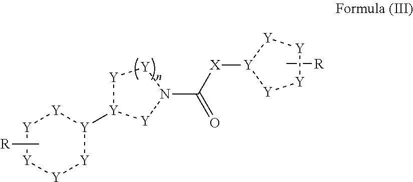

wherein:
each ---- independently represents a single bond or a double bond; X=CH₂, CHR, CR₂, or CH═CH;
each Y is independently CR^{a}R^{b}, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

In some embodiments, the FabI inhibitor is a compound according to Formula (IV):

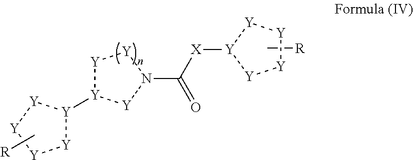

wherein:
each ---- independently represents a single bond or a double bond; X=CH₂, CHR, CR₂, or CH═CH;
each Y is independently CR^{a}R^{b}, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

In some embodiments, the

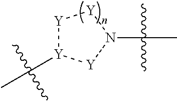

moiety of the compound of Formulas (I)-(IV) is selected from the group consisting of:

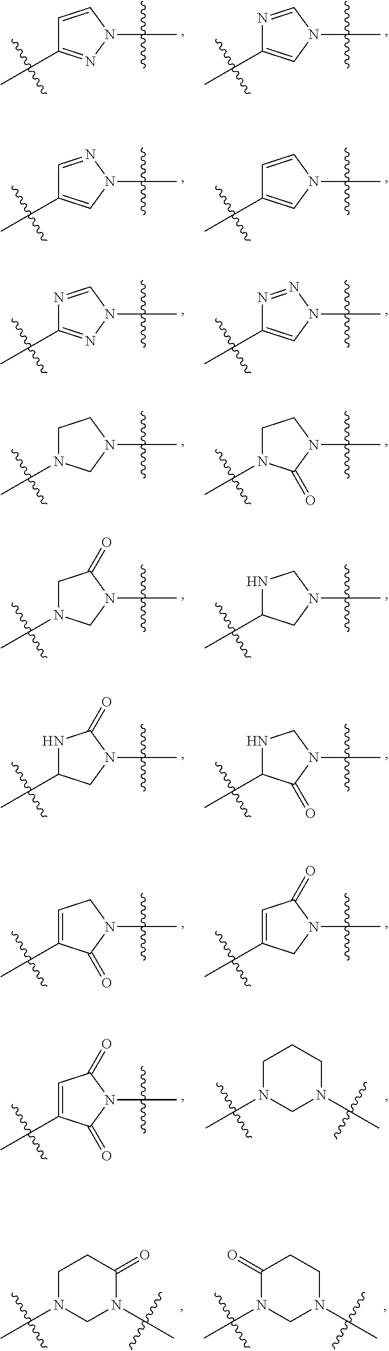

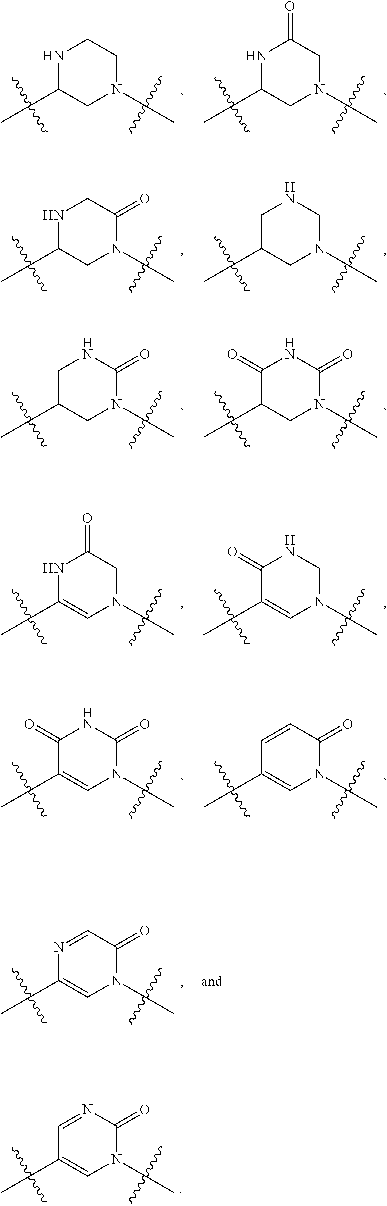

In some embodiments, the FabI inhibitor is one or more compound selected from Formulas (Ia) to (IVp):

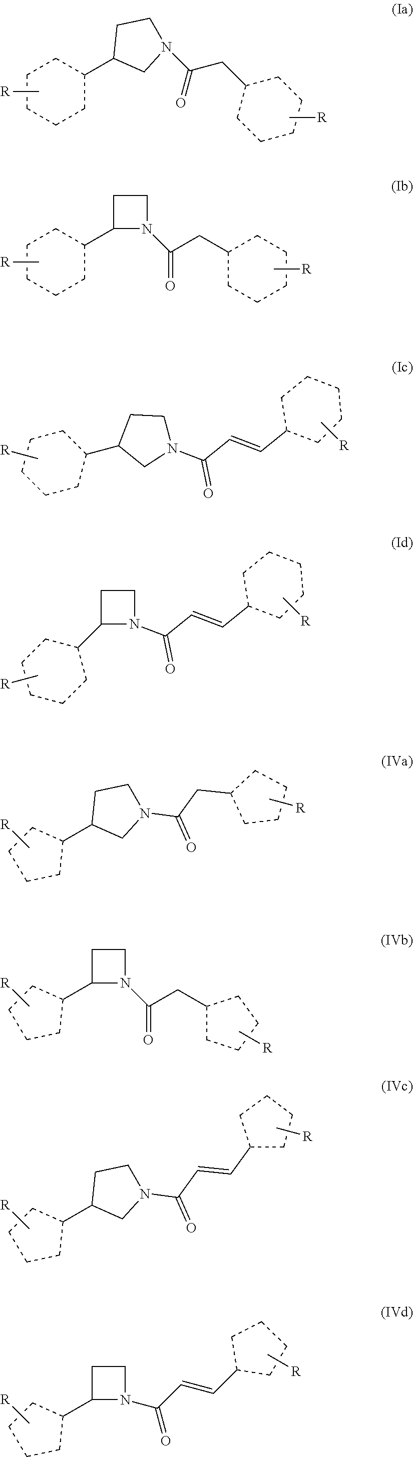

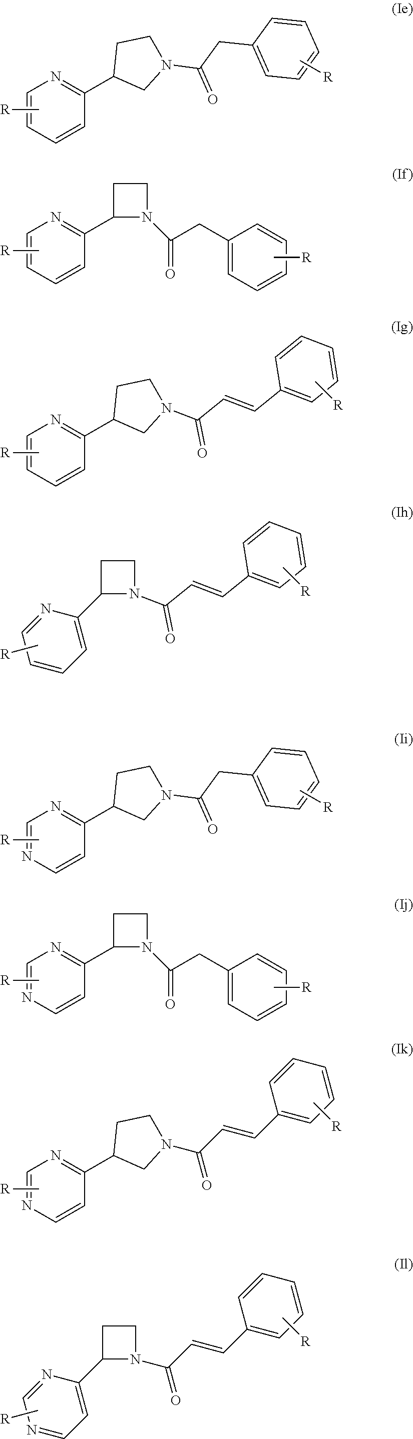

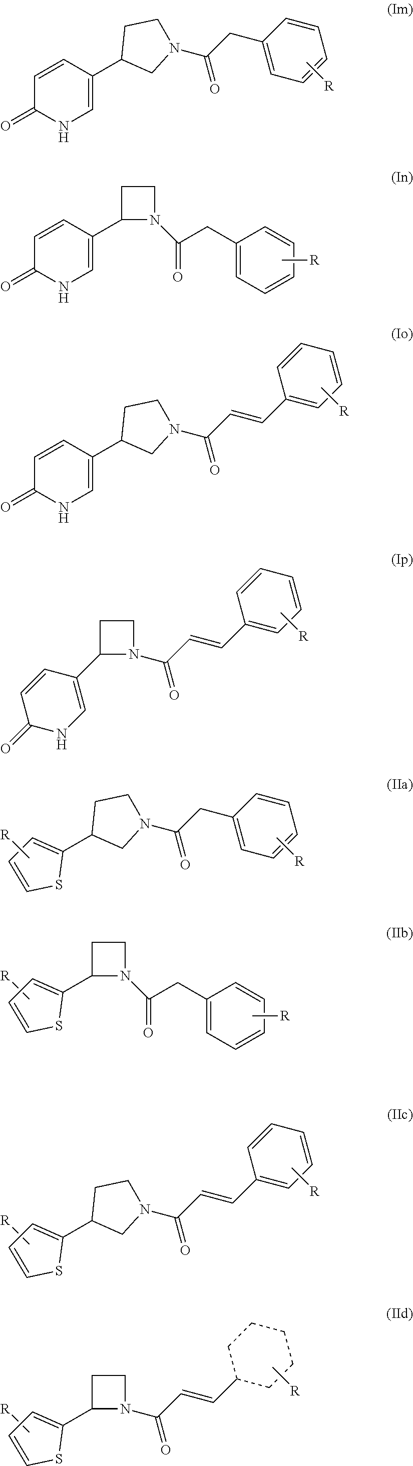

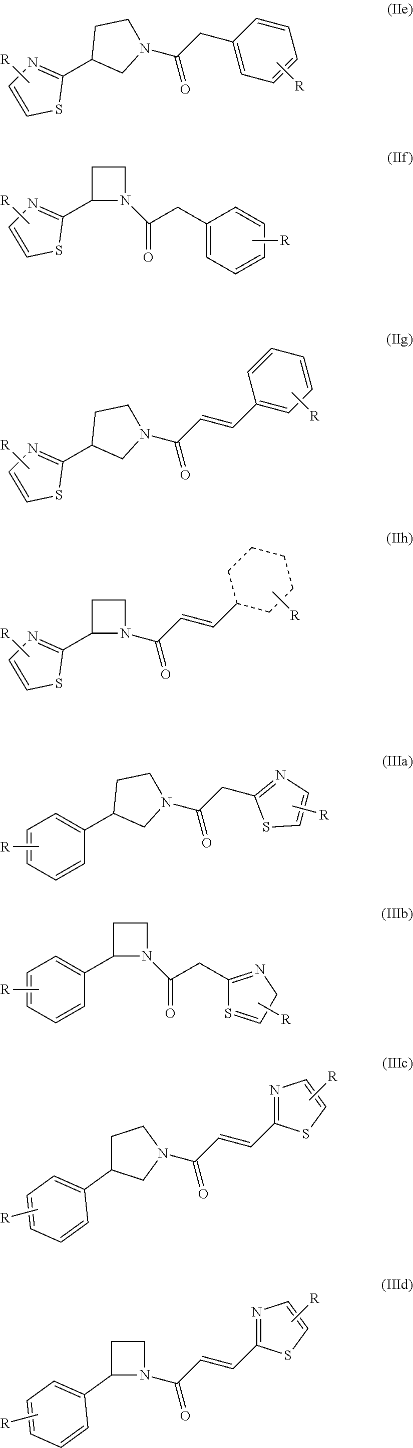

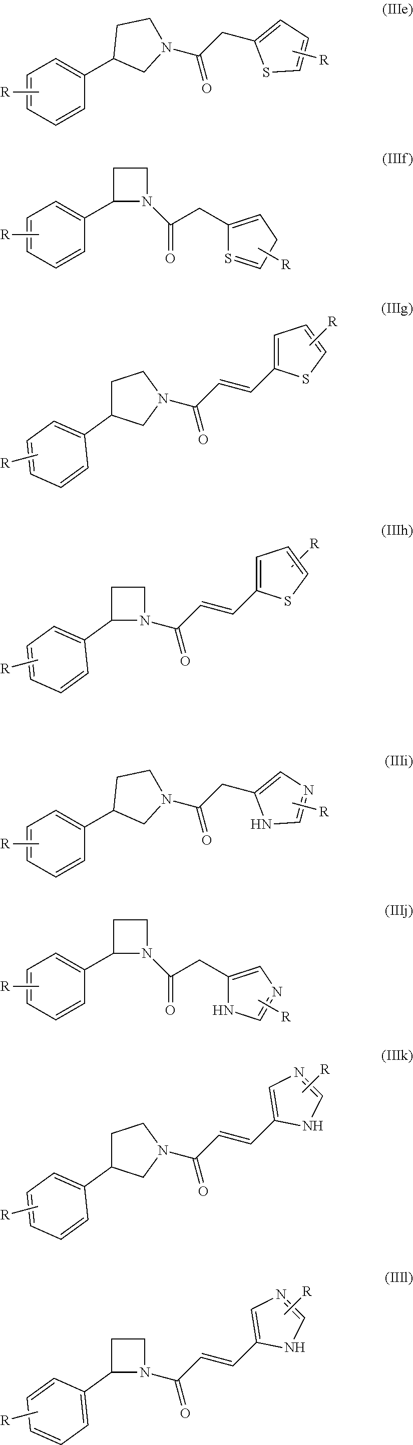

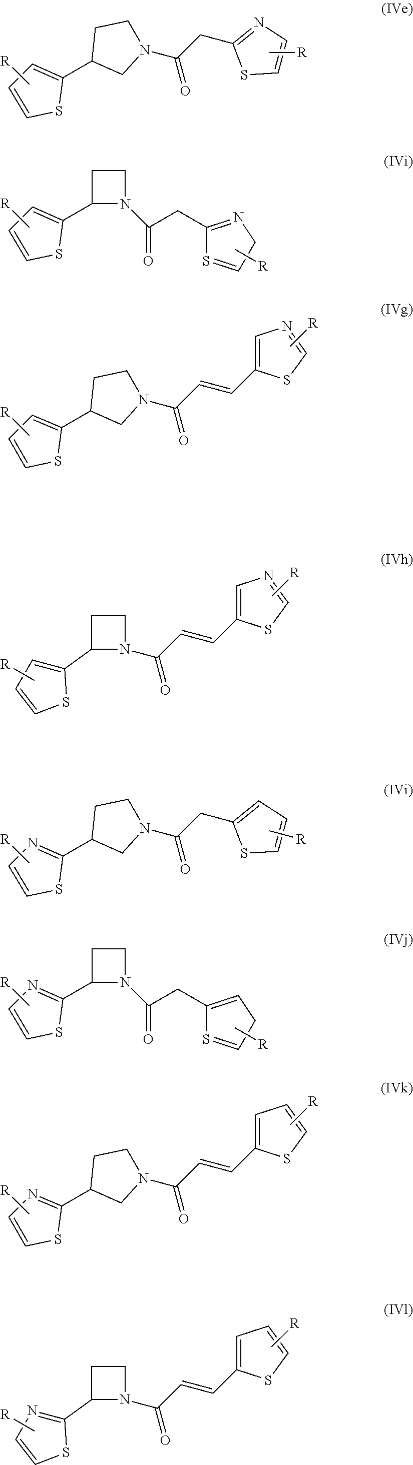

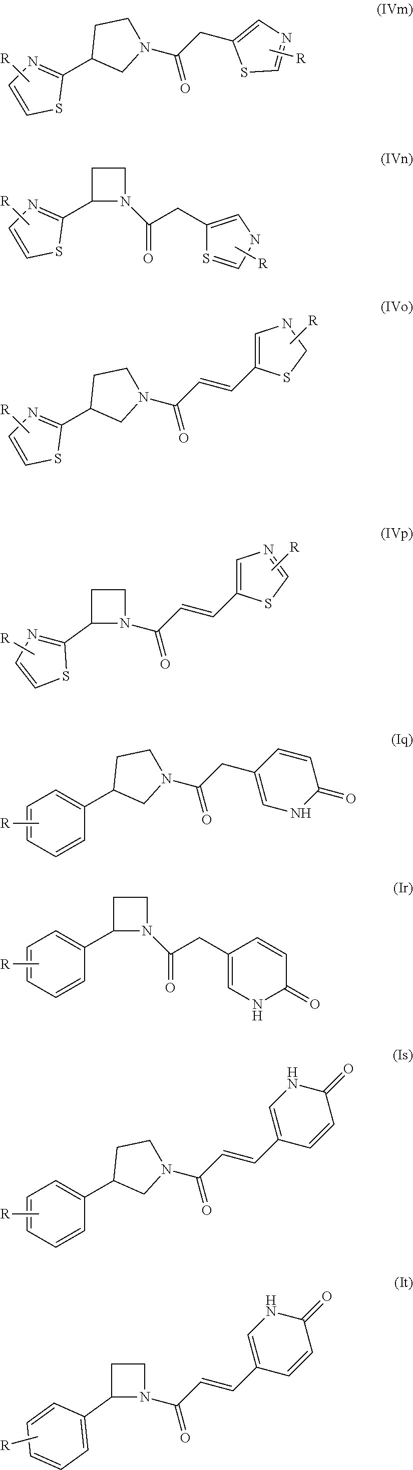

In some embodiments, the FabI inhibitor is a tautomer, polymorph, stereoisomer, prodrug, solvate, or pharmaceutically acceptable salt of a compound according to any one of Formulas (I)-(IV), for example a tautomer, polymorph, stereoisomer, prodrug, solvate, or pharmaceutically acceptable salt of a compound according to any one of Formulas (Ia)-(IVp).

A suitable tautomer, polymorph, stereoisomer, prodrug, solvate, or pharmaceutically acceptable salt may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

In some embodiments, the present disclosure provides a pharmaceutical composition comprising one or more FabI inhibitors disclosed herein. In some embodiments, the pharmaceutical composition comprises at least one (e.g., one, two, three, four, or more than four) FabI inhibitors as disclosed herein.

In some embodiments, the pharmaceutical composition comprises a compound according to Formula (I), such as a compound according to any one of Formulas (Ia)-(It). In another embodiment, the pharmaceutical composition comprises two compounds according to Formula (I), such as two compounds according to any one of Formulas (Ia)-(It). In another embodiment, the pharmaceutical composition comprises three compounds according to Formula (I), such as three compounds according to any one of Formulas (Ia)-(It). In another embodiment, the pharmaceutical composition comprises four compounds according to Formula (I), such as four compounds according to any one of Formulas (Ia)-(It).

In some embodiments, the pharmaceutical composition comprises a compound according to Formula (II), such as a compound according to any one of Formulas (IIa)-(IIh). In another embodiment, the pharmaceutical composition comprises two compounds according to Formula (II), such as two compounds according to any one of Formulas (IIa)-(IIh). In another embodiment, the pharmaceutical composition comprises three compounds according to Formula (II), such as three compounds according to any one of Formulas (IIa)-(IIh). In another embodiment, the pharmaceutical composition comprises four compounds according to Formula (II), such as four compounds according to any one of Formulas (IIa)-(IIh).

In some embodiments, the pharmaceutical composition comprises a compound according to Formula (III), such as a compound according to any one of Formulas (IIIa)-(IIIl). In another embodiment, the pharmaceutical composition comprises two compounds according to Formula (III), such as two compounds according to any one of Formulas (IIIa)-(IIIl). In another embodiment, the pharmaceutical composition comprises three compounds according to Formula (III), such as three compounds according to any one of Formulas (IIIa)-(IIIl). In another embodiment, the pharmaceutical composition comprises four compounds according to Formula (III), such as four compounds according to any one of Formulas (IIIa)-(IIIl).

In some embodiments, the pharmaceutical composition comprises a compound according to Formula (IV), such as a compound according to any one of Formulas (IVa)-(IVp). In another embodiment, the pharmaceutical composition comprises two compounds according to Formula (IV), such as two compounds according to any one of Formulas (IVa)-(IVp). In another embodiment, the pharmaceutical composition comprises three compounds according to Formula (IV), such as three compounds according to any one of Formulas (IVa)-(IVp). In another embodiment, the pharmaceutical composition comprises four compounds according to Formula (IV), such as four compounds according to any one of Formulas (IVa)-(IVp).

In some embodiments, the pharmaceutical composition comprises a compound according to Formula (I) and a compound according to any one of Formulas (II)-(IV). In some embodiments, the pharmaceutical composition comprises a compound according to Formula (I) and two compounds according to Formulas (II)-(IV). In some embodiments, the pharmaceutical composition comprises a compound according to Formula (I) and three compounds according to Formulas (II)-(IV).

In some embodiments, the pharmaceutical composition comprises a compound according to Formula (II) and a compound according to any one of Formulas (I) or (III)-(IV). In some embodiments, the pharmaceutical composition comprises a compound according to Formula (II) and two compounds according to Formulas (I) or (III)-(IV). In some embodiments, the pharmaceutical composition comprises a compound according to Formula (II) and three compounds according to Formulas (I) or (III)-(IV).

In some embodiments, the pharmaceutical composition comprises a compound according to Formula (III) and a compound according to any one of Formulas (I)-(II) or (IV). In some embodiments, the pharmaceutical composition comprises a compound according to Formula (III) and two compounds according to Formulas (I)-(II) or (IV). In some embodiments, the pharmaceutical composition comprises a compound according to Formula (III) and three compounds according to Formulas (I)-(II) or (IV).

In some embodiments, the pharmaceutical composition comprises a compound according to Formula (IV) and a compound according to any one of Formulas (I)-(III). In some embodiments, the pharmaceutical composition comprises a compound according to Formula (IV) and two compounds according to Formulas (I)-(III). In some embodiments, the pharmaceutical composition comprises a compound according to Formula (IV) and three compounds according to Formulas (I)-(III).

In some embodiments, a pharmaceutical composition of the present disclosure comprises a FabI inhibitor in an amount (or a combination of more than one FabI inhibitor in a total amount) of about 5 mg to about 5000 mg, for example about 50 mg to about 2500 mg, about 100 mg to about 1000 mg, about 250 mg to about 500 mg, such as example about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

Pharmaceutical compositions of the invention optionally comprise one or more pharmaceutically acceptable excipients. The term “pharmaceutically acceptable excipient” herein means any substance, not itself a therapeutic agent, used as a carrier or vehicle for delivery of a therapeutic agent (e.g., a FabI inhibitor) to a subject or added to a pharmaceutical composition to improve its handling or storage properties or to permit or facilitate formation of a unit dose of the composition, and that does not produce unacceptable toxicity or interaction with other components in the pharmaceutical composition.

In some embodiments, a pharmaceutical composition according to the present disclosure comprises a pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of a- and amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the pharmaceutical composition.

In some embodiments, a pharmaceutical composition according to the present disclosure comprises one or more pharmaceutically acceptable disintegrants as excipients. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums. Such disintegrants, if present, typically comprise in total about 0.2% to about 30%, about 0.2% to about 10%, or about 0.2% to about 5%, of the total weight of the pharmaceutical composition.

In some embodiments, a pharmaceutical composition according to the present disclosure comprises one or more antioxidants. Illustrative antioxidants include sodium ascorbate and vitamin E (tocopherol). One or more antioxidants, if present, are typically present in a pharmaceutical composition of the invention in an amount of about 0.001% to about 5%, about 0.005% to about 2.5%, or about 0.01% to about 1%, by weight.

In some embodiments, a pharmaceutical composition according to the present disclosure comprises one or more pharmaceutically acceptable binding agents or adhesives as excipients. Such binding agents and adhesives can impart sufficient cohesion to a powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the pharmaceutical composition.

In some embodiments, a pharmaceutical composition according to the present disclosure comprises one or more pharmaceutically acceptable wetting agents as excipients. Non-limiting examples of surfactants that can be used as wetting agents in pharmaceutical compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof Such wetting agents, if present, constitute in total about 0.25% to about 15%, about 0.4% to about 10%, or about 0.5% to about 5%, of the total weight of the pharmaceutical composition.

In some embodiments, a pharmaceutical composition according to the present disclosure comprises one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the pharmaceutical composition.

Suitable anti-adherents include talc, cornstarch, DL-leucine, sodium lauryl sulfate and metallic stearates. Talc is an anti-adherent or glidant used, for example, to reduce formulation sticking to equipment surfaces and also to reduce static in the blend. Talc, if present, constitutes about 0.1% to about 10%, about 0.25% to about 5%, or about 0.5% to about 2%, of the total weight of the pharmaceutical composition. Glidants can be used to promote powder flow of a solid formulation. Suitable glidants include colloidal silicon dioxide, starch, talc, tribasic calcium phosphate, powdered cellulose and magnesium trisilicate.

In some embodiments, a pharmaceutical composition according to the present disclosure comprises one or more flavoring agents, sweetening agents, and/or colorants. Flavoring agents useful in the present invention include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents that can be used in a pharmaceutical composition according to the present disclosure include, for example, acesulfame potassium (acesulfame K), alitame, aspartame, cyclamate, cylamate, dextrose, isomalt, MagnaSweet®, maltitol, mannitol, neohesperidine DC, neotame, Prosweet® Powder, saccharin, sorbitol, stevia, sucralose, sucrose, tagatose, thaumatin, xylitol, and the like.

Flavoring agents, sweetening agents, and/or colorants can be present in a pharmaceutical composition of the present disclosure in any suitable amount, for example about 0.01% to about 10%, about 0.1% to about 8%, or about 1% to about 5%, by weight of the pharmaceutical composition.

In some embodiments, a pharmaceutical composition according to the present disclosure comprises a suspending agent. Non-limiting illustrative examples of suitable suspending agents include silicon dioxide, bentonite, hydrated aluminum silicate (e.g. kaolin) and mixtures thereof. The suspending agent(s), if present, may comprise a total amount of about 0.01% to about 3.0%, about 0.1% to about 2.0%, or about 0.25% to about 1.0%, by weight of the pharmaceutical composition.

The foregoing excipients can have multiple roles as is known in the art. For example, starch can serve as a filler as well as a disintegrant. The classification of excipients above is not to be construed as limiting in any manner. Excipients categorized in any manner may also operate under various different categories of excipients as will be readily appreciated by one of ordinary skill in the art.

In various embodiments, compounds and pharmaceutical compositions of the invention are useful for treatment and/or prevention of a disease or disorder. As used herein, the term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. As used herein, the term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In some embodiments, the present disclosure provides a method of treating or preventing a disease or disorder in a subject, the method comprising administering to the subject an effective amount of at least one FabI inhibitor as disclosed herein, or a pharmaceutical composition comprising at least one FabI inhibitor as disclosed herein. In some embodiments, the at least one FabI inhibitor has a structure according to any one of Formulas (I)-(IV), for example any one of Formulas (Ia)-(IVp). In some embodiments, the at least one FabI inhibitor is selected from the group consisting of:

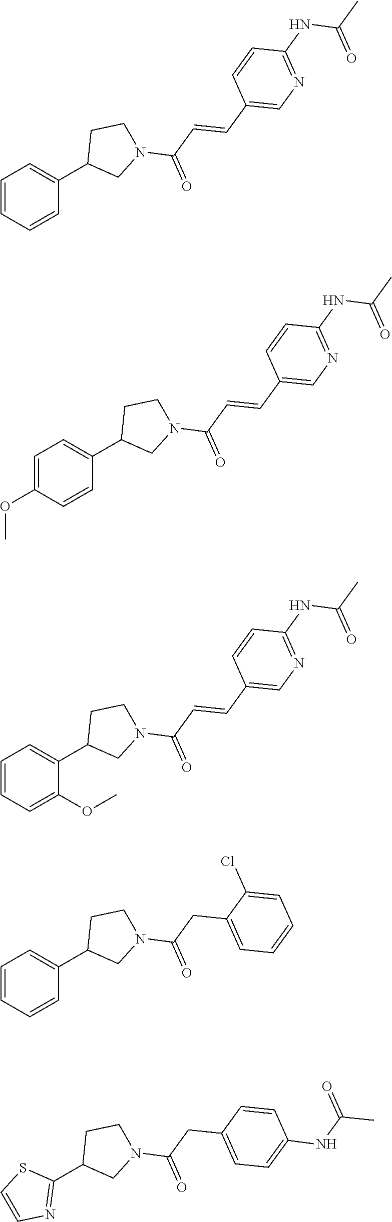 [SMILES: CC(=O)Nc1ccc(/C=C/C(=O)N2CCC(c3ccccc3)C2)cn1 ; COc1ccc(C2CCN(C(=O)/C=C/c3ccc(NC(C)=O)nc3)C2)cc1 ; COc1ccccc1C1CCN(C(=O)/C=C/c2ccc(NC(C)=O)nc2)C1 ; O=C(Cc1ccccc1Cl)N1CCC(c2ccccc2)C1 ; CC(=O)Nc1ccc(CC(=O)N2CCC(c3nccs3)C2)cc1]

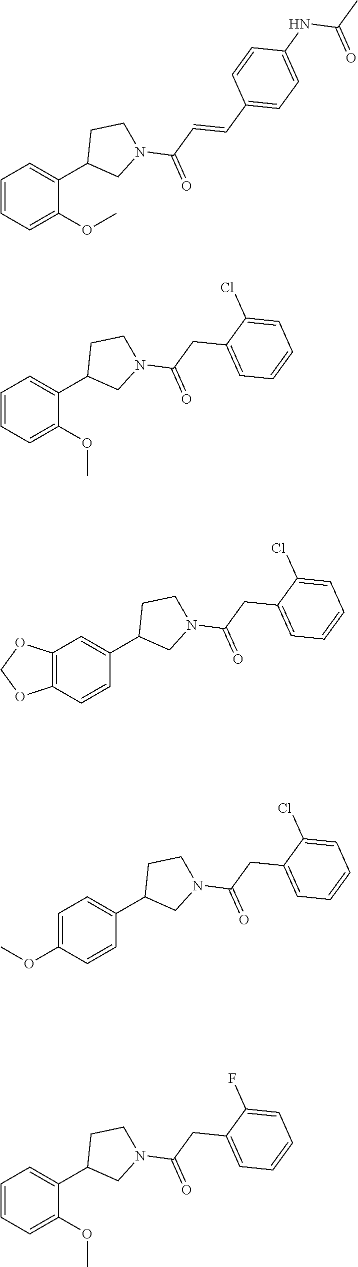

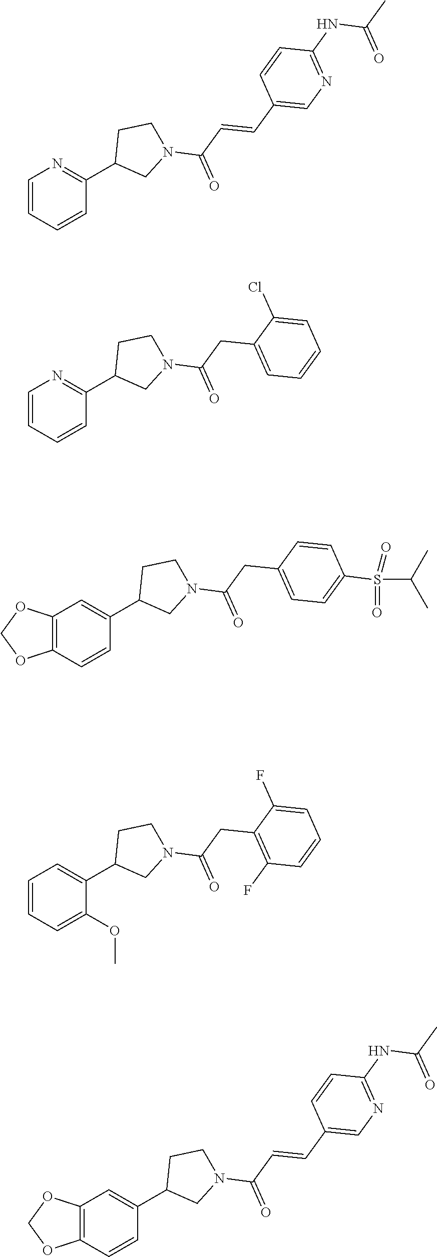 [SMILES: CC(=O)Nc1ccc(/C=C/C(=O)N2CCC(c3ccccn3)C2)cn1 ; O=C(Cc1ccccc1Cl)N1CCC(c2ccccn2)C1 ; CC(C)S(=O)(=O)c1ccc(CC(=O)N2CCC(c3ccc4c(c3)OCO4)C2)cc1 ; COc1ccccc1C1CCN(C(=O)Cc2c(F)cccc2F)C1 ; CC(=O)Nc1ccc(/C=C/C(=O)N2CCC(c3ccc4c(c3)OCO4)C2)cn1]

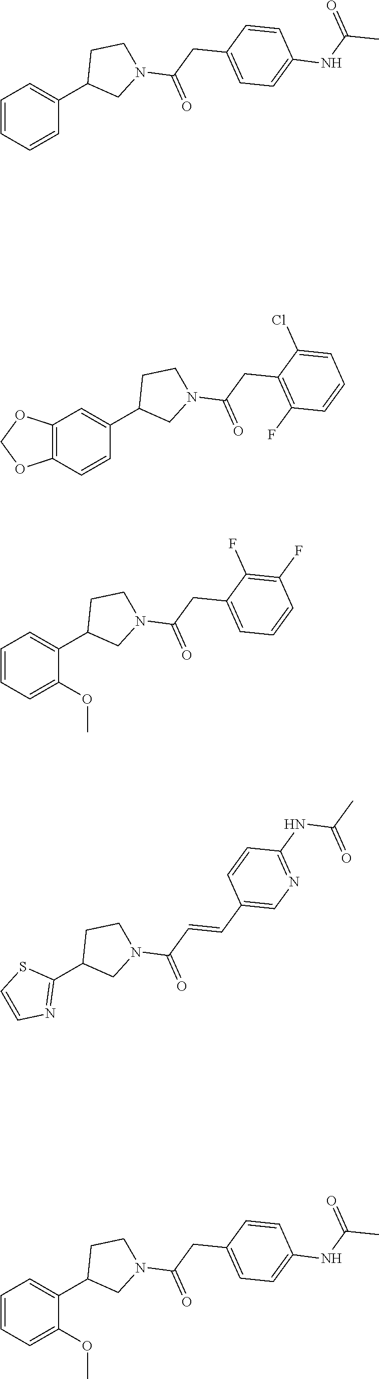 [SMILES: CC(=O)Nc1ccc(CC(=O)N2CCC(c3ccccc3)C2)cc1 ; O=C(Cc1c(F)cccc1Cl)N1CCC(c2ccc3c(c2)OCO3)C1 ; COc1ccccc1C1CCN(C(=O)Cc2cccc(F)c2F)C1 ; CC(=O)Nc1ccc(/C=C/C(=O)N2CCC(c3nccs3)C2)cn1 ; COc1ccccc1C1CCN(C(=O)Cc2ccc(NC(C)=O)cc2)C1]

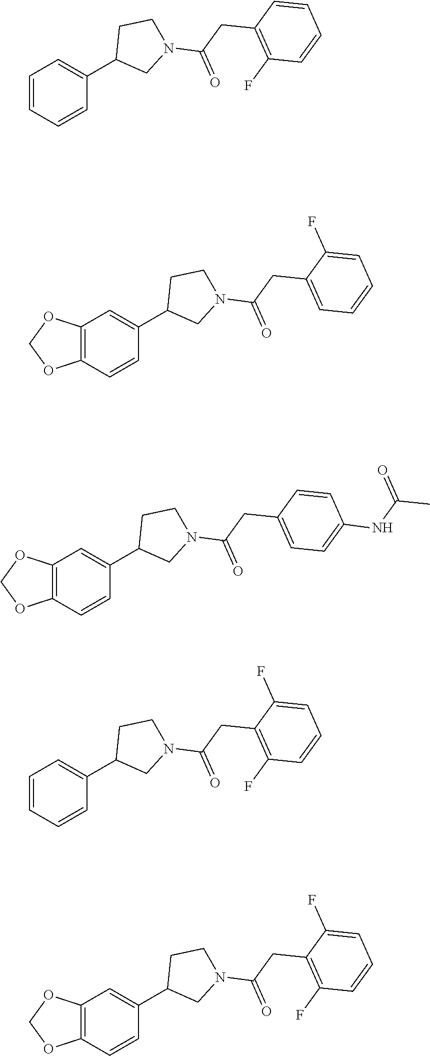 [SMILES: O=C(Cc1ccccc1F)N1CCC(c2ccccc2)C1 ; O=C(Cc1ccccc1F)N1CCC(c2ccc3c(c2)OCO3)C1 ; CC(=O)Nc1ccc(CC(=O)N2CCC(c3ccc4c(c3)OCO4)C2)cc1 ; O=C(Cc1c(F)cccc1F)N1CCC(c2ccccc2)C1 ; O=C(Cc1c(F)cccc1F)N1CCC(c2ccc3c(c2)OCO3)C1]

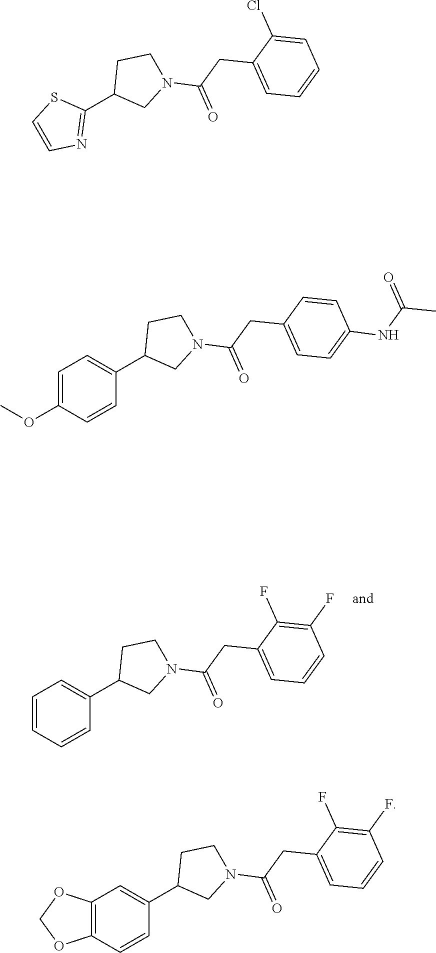 [SMILES: O=C(Cc1ccccc1Cl)N1CCC(c2nccs2)C1 ; COc1ccc(C2CCN(C(=O)Cc3ccc(NC(C)=O)cc3)C2)cc1 ; O=C(Cc1cccc(F)c1F)N1CCC(c2ccccc2)C1 ; O=C(Cc1cccc(F)c1F)N1CCC(c2ccc3c(c2)OCO3)C1]

In some embodiments, the disease or disorder is associated with an organism that utilizes FabI as its primary enoyl reductase. In some embodiments, the organism is one or more of: Francisella tularensis, Staphylococcus aureus, Bacillus anthraces, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsiarickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia coli, Haemophilus influenzae, Helicobacter pylori, Klebsiella pneumoniae, and Neisseria meningitidis.

In some embodiments, the method further comprises identifying the subject has having a disease or disorder associated with an organism that utilizes FabI as its primary enoyl reductase before the step of administering the compound or pharmaceutical composition. In some embodiments, the disease or disorder is one or more of: a skin infection, an infection of a surgical wound, an infection of a trauma wound, a urinary tract infection, food poisoning, a gastrointestinal tract infection, an infection of an organ, pneumonia (a lung infection), osteomyelitis (a bone infection), endocarditis (a heart infection), phlebitis (an infection of veins and blood vessels), mastitis (an infection of breast and formation of abscesses), meningitis (a brain infection), an infection from and/or on an indwelling medical device (e.g., a joint prosthesis, a cardiovascular device, an artificial heart valve, etc.), a blood infection (e.g., a generalized life threatening blood infection), Toxic shock syndrome (TSS), bacteremia, septicemia, an intra-abdominal infection, a pelvic infection, a soft tissue infection, bacteremia, endocarditis, Anthrax, plague, malaria, a food borne illnesses, food poisoning, whooping cough, brucellosis, brucellosis in a canine, brucellosis in a livestock animal, brucellosis in a swine, camplyobacteriosis, an ulcer, a stomach infection, Legionnaires disease, gonorrhea, meningococcal disease, rickettsial (spotted and/or typhus fevers) and related infections (e.g., anaplasmosis and ehrlichiosis), gastroenteritis, enteric fever, shigellosis, cholera, chlamydia, avian chlamydiosis, tuberculosis, leprosy, ulcerative skin disease, pharyngitis, bronchitis, coronary artery disease, atypical pneumonia, conjunctivitis, endocarditis, septic arthritis, CNS infections, ophthalmic infections, and listeriosis.

In one embodiment, the method comprises administering to the subject a FabI inhibitor having a structure according to any one of Formulas (I)-(IV), such as any one of Formulas (Ia)-(IVp), or a pharmaceutical composition comprising at least one FabI inhibitor having a structure according to any one of Formulas (I)-(IV), such as any one of Formulas (Ia)-(IVp), in an amount sufficient to provide a daily dose of the FabI inhibitor(s) of about 1 mg to about 10,000 mg, for example about 5 mg to about 5000 mg, about 50 mg to about 2500 mg, about 100 mg to about 1000 mg, or about 250 mg to about 500 mg, such as about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2750 mg, about 3000 mg, about 3250 mg, about 3500 mg, about 3750 mg, about 3800 mg, about 4000 mg, about 4250 mg, about 4500 mg, about 4750 mg, about 5000 mg, about 5500 mg, about 6000 mg, about 6500 mg, about 7000 mg, about 7500 mg, about 8000 mg, about 8500 mg, about 9000 mg, about 9500 mg, or about 10,000 mg.

In some embodiments, the method further comprises discontinuing an existing therapeutic regimen in the subject and thereafter administering a FabI inhibitor according to the present disclosure, or a pharmaceutical composition comprising at least one FabI inhibitor according to the present disclosure.

In some embodiments, the present disclosure provides a FabI inhibitor for use in the manufacture of a medicament for treating or preventing a disease or disorder associated with an organism that utilizes FabI as its primary enoyl reductase. In some embodiments, the organism is one or more of: Francisella tularensis, Staphylococcus aureus, Bacillus anthraces, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsiarickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia coli, Haemophilus influenzae, Helicobacter pylori, Klebsiella pneumoniae, and Neisseria meningitidis.

In some embodiments, the method further comprises identifying the subject has having a disease or disorder associated with an organism that utilizes FabI as its primary enoyl reductase before the step of administering the medicament to the subject. In some embodiments, the disease or disorder is one or more of: a skin infection, an infection of a surgical wound, an infection of a trauma wound, a urinary tract infection, food poisoning, a gastrointestinal tract infection, an infection of an organ, pneumonia (a lung infection), osteomyelitis (a bone infection), endocarditis (a heart infection), phlebitis (an infection of veins and blood vessels), mastitis (an infection of breast and formation of abscesses), meningitis (a brain infection), an infection from and/or on an indwelling medical device (e.g., a joint prosthesis, a cardiovascular device, an artificial heart valve, etc.), a blood infection (e.g., a generalized life threatening blood infection), Toxic shock syndrome (TSS), bacteremia, septicemia, an intra-abdominal infection, a pelvic infection, a soft tissue infection, bacteremia, endocarditis, Anthrax, plague, malaria, a food borne illnesses, food poisoning, whooping cough, brucellosis, brucellosis in a canine, brucellosis in a livestock animal, brucellosis in a swine, camplyobacteriosis, an ulcer, a stomach infection, Legionnaires' disease, gonorrhea, meningococcal disease, rickettsial (spotted and/or typhus fevers) and related infections (e.g., anaplasmosis and ehrlichiosis), gastroenteritis, enteric fever, shigellosis, cholera, chlamydia, avian chlamydiosis, tuberculosis, leprosy, ulcerative skin disease, pharyngitis, bronchitis, coronary artery disease, atypical pneumonia, conjunctivitis, endocarditis, septic arthritis, CNS infections, ophthalmic infections, and listeriosis.

In some embodiments, the medicament comprises a FabI inhibitor in an amount (or a combination of more than one FabI inhibitor in a total amount) of about 5 mg to about 5000 mg, for example about 50 mg to about 2500 mg, about 100 mg to about 1000 mg, about 250 mg to about 500 mg, such as example about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg. In some embodiments, the at least one FabI inhibitor has a structure according to any one of Formulas (I)-(IV), for example any one of Formulas (Ia)-(IVp) and/or Table 1.

While the technology of the present disclosure is capable of being embodied in various forms, the description of several embodiments herein is made with the understanding that the present disclosure is to be considered as an exemplification of the inventive technology, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

### Example

Examples of representative compounds of Formulas (I)-(IV) are shown in Table 1 below, along with determined enzyme inhibition (IC₅₀) levels and antimicrobial activities against five bacteria.

IC₅₀ Determinations for Both Staphylococcus aureus FabI (SaFabI) and Acinetobacter baumannii FabI (AbFabI):

Buffer condition: Reactions were carried out in 50 mM MES buffer at pH 5.5 with 100 mM NaCl, 0.1 mg/ml BSA and 0.01% triton and 200 nM enzyme in a final volume of 50 μl in a 384-well plate format.

Procedure: The assay was conducted using the above-mentioned buffer with 300 μM crotonyl-coenzymeA and 200 μM NADPH for SafabI, while 200 μM NADH was used for AbFabI. Reaction was monitored by following the decrease NADPH or NADH fluorescence (excitation at 340 nm and emission at 460 nm) during the reaction as NADPH or NADH is oxidised to NADP+ or NAD+. Compound concentrations were varied from 200 μM to 0.4 nM. The compounds were incubated with the enzyme in the well for 20 minutes before addition of crotonyl-coenzymeA. Linear slopes for the first ten minutes were used to determine reaction rate. The percent inhibition was plotted as a function of inhibitor concentration and the data fit via nonlinear regression to the Hill equation

y=Vmax*xⁿ/(IC₅₀ⁿ+xⁿ),

where Vmax.=maximum inhibition, and n=Hill coefficient.

MIC Determinations:

The minimum inhibitory concentrations were tested against the two S. aureus strains (strain RN4220 and strain Newman), F. tularensis (strain Utah), E. coli (strain BW25113) and E. coli tolC- (strain BW25113, efflux pump mutant). Optimal growth media (TSB media for S. aureus and LB media with F. tularensis and the two E. coli strains) was added to each well in a row on a sterile 96-well flat bottom tissue culture plate. 96 μL of media was added to the first column and 50 μL was added to all subsequent wells. The compounds to be tested were added to the first column for a final well volume of 100 μL. These compounds were then serially diluted (2-fold) across the columns of wells by pipetting and mixing 50 μl of solution. The extra 50 μl was discarded from the final wells. Triclosan and erythromycin were used as controls in these studies. Prior to setting up the MIC plates, the appropriate bacterial cultures were grown to mid log-phase and subsequently diluted with fresh media. The OD600 of the diluted culture was 0.004 for E. coli and E. tularensis while it was 0.0001 for S. aureus. 04. Then 50 μl of this culture was added to each well of the plate and the plate was then incubated at 37° C. overnight without shaking. For each compound, the first clear well with no signs of visible growth was reported as the MIC value. The MIC values are reported in units of μg/mL.

TABLE 1

| | Enzyme | | Enzyme | | | | | | |
|---|---|---|---|---|---|---|---|---|---|
| | inhibition | | inhibition | | MIC (μg/mL) | | | | |
| | or | | or | | S. aureus | S. aureus | E. coli | | |
| | IC50 with | | IC50 with | | RN4220 | Newman | BW25113 | E. coli | F. tularensis |
| Structure | SaFabl | | AbFabl | | WT | WT | WT | TolC- | strain Utah |
| 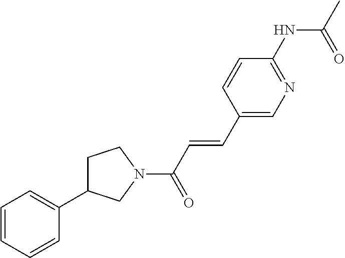 [SMILES: CC(=O)Nc1ccc(/C=C/C(=O)N2CCC(c3ccccc3)C2)cn1] | 1.2 | μM | 0.47 | μM | >200 | >200 | >200 | 12.5 | >200 |
| 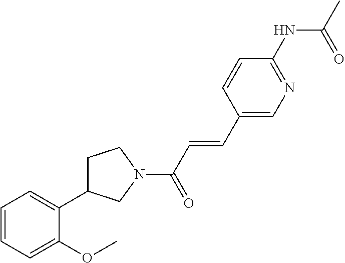 [SMILES: COc1ccccc1C1CCN(C(=O)/C=C/c2ccc(NC(C)=O)nc2)C1] | 3.5 | μM | 0.42 | μM | 12.5-25 | 12.5-25 | >200 | 0.75 | 12.5 |
| 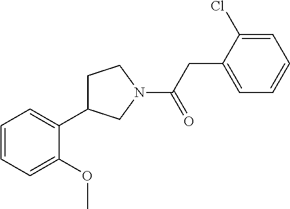 [SMILES: COc1ccccc1C1CCN(C(=O)Cc2ccccc2Cl)C1] | 0.5 | μM | 0.88 | μM | 12.5-25 | 25 | >200 | >200 | 50 |
| 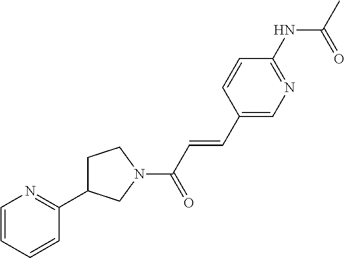 [SMILES: CC(=O)Nc1ccc(/C=C/C(=O)N2CCC(c3ccccn3)C2)cn1] | 28.4 | μM | 5.0 | μM | >100 | NT | >100 | 100 | >100 |
| 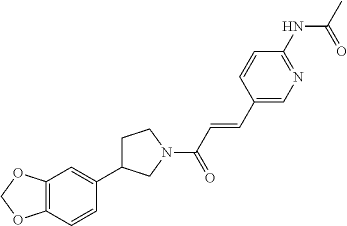 [SMILES: CC(=O)Nc1ccc(/C=C/C(=O)N2CCC(c3ccc4c(c3)OCO4)C2)cn1] | 1.1 | μM | 8.5 | μM | >200 | >200 | >100 | >100 | >200 |
| 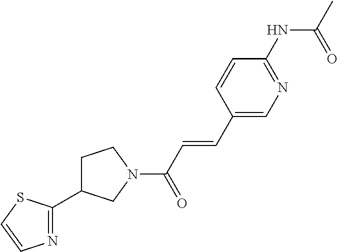 [SMILES: CC(=O)Nc1ccc(/C=C/C(=O)N2CCC(c3nccs3)C2)cn1] | 4.5 | μM | 10-20 | μM | >100 | NT | >100 | >100 | >100 |
| 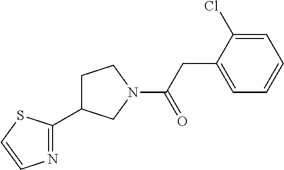 [SMILES: O=C(Cc1ccccc1Cl)N1CCC(c2nccs2)C1] | 6 | μM | 14 | μM | 50 | 50 | >100 | >100 | 200 |
| 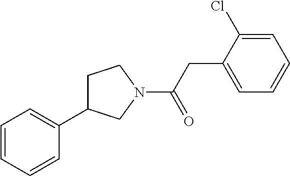 [SMILES: O=C(Cc1ccccc1Cl)N1CCC(c2ccccc2)C1] | 10.7 | μM | 3.1 | μM | 12.5 | 12.5-25 | >200 | 50 | 25 |
| 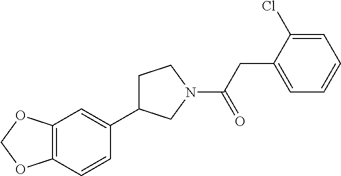 [SMILES: O=C(Cc1ccccc1Cl)N1CCC(c2ccc3c(c2)OCO3)C1] | 2.5 | μM | 3.7 | μM | 12.5 | 6.25-12.5 | >200 | 25 | 25 |
| 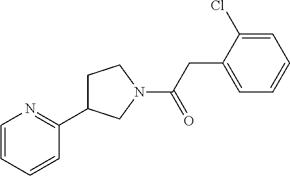 [SMILES: O=C(Cc1ccccc1Cl)N1CCC(c2ccccn2)C1] | 18.4 | μM | 30.7 | μM | 100 | 50 | >200 | >200 | 200 |
| 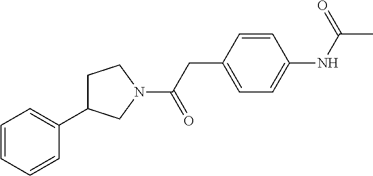 [SMILES: CC(=O)Nc1ccc(CC(=O)N2CCC(c3ccccc3)C2)cc1] | >100 | μM | >100 | μM | >200 | >200 | >200 | 200 | 200 |
| 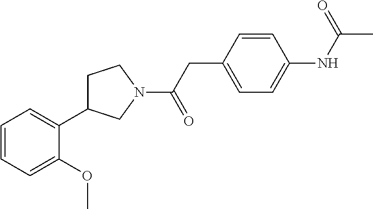 [SMILES: COc1ccccc1C1CCN(C(=O)Cc2ccc(NC(C)=O)cc2)C1] | >100 | μM | >100 | μM | >200 | >200 | >200 | >200 | >200 |
| 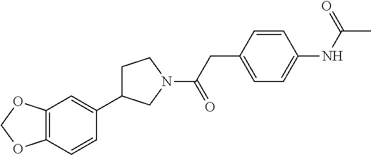 [SMILES: CC(=O)Nc1ccc(CC(=O)N2CCC(c3ccc4c(c3)OCO4)C2)cc1] | >100 | μM | >100 | μM | >200 | >200 | >200 | >200 | >200 |
| 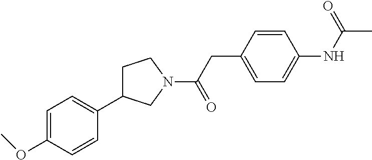 [SMILES: COc1ccc(C2CCN(C(=O)Cc3ccc(NC(C)=O)cc3)C2)cc1] | >100 | μM | >100 | μM | >200 | >200 | >200 | >200 | >200 |
| 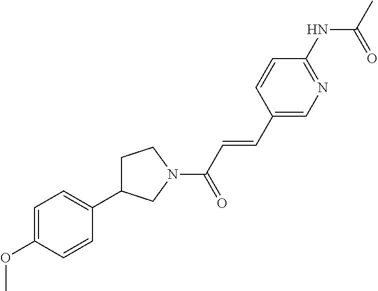 [SMILES: COc1ccc(C2CCN(C(=O)/C=C/c3ccc(NC(C)=O)nc3)C2)cc1] | 3.55 | μM | >100 | μM | >200 | >200 | >200 | >200 | >200 |
| 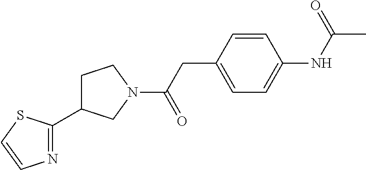 [SMILES: CC(=O)Nc1ccc(CC(=O)N2CCC(c3nccs3)C2)cc1] | >100 | μM | >100 | μM | >200 | >200 | >200 | >200 | >200 |
| 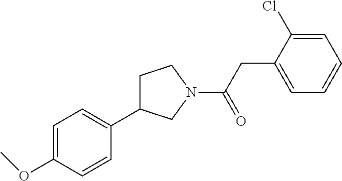 [SMILES: COc1ccc(C2CCN(C(=O)Cc3ccccc3Cl)C2)cc1] | 12.5 | μM | >200 | μM | 100 | >200 | >200 | 100 | 12.5 |
| 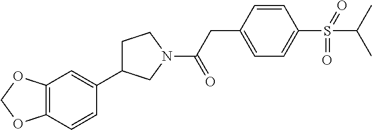 [SMILES: CC(C)S(=O)(=O)c1ccc(CC(=O)N2CCC(c3ccc4c(c3)OCO4)C2)cc1] | >100 | μM | >100 | μM | >200 | >200 | >200 | >200 | 100 |
| 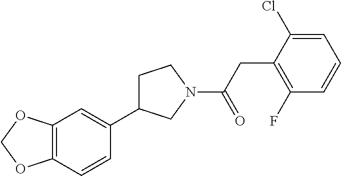 [SMILES: O=C(Cc1c(F)cccc1Cl)N1CCC(c2ccc3c(c2)OCO3)C1] | 1.8 | μM | >100 | μM | 25 | 25 | >200 | >200 | 25 |
| 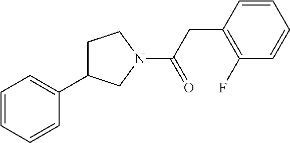 [SMILES: O=C(Cc1ccccc1F)N1CCC(c2ccccc2)C1] | 5.0 | μM | 2.5 | μM | 50 | 50-100 | >200 | >200 | 50 |
| 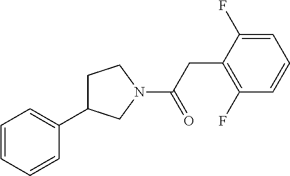 [SMILES: O=C(Cc1c(F)cccc1F)N1CCC(c2ccccc2)C1] | 1.9 | μM | 6.9 | μM | 50 | 50-100 | >200 | >200 | 50 |
| 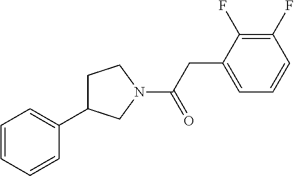 [SMILES: O=C(Cc1cccc(F)c1F)N1CCC(c2ccccc2)C1] | 7.3 | μM | 9.3 | μM | 50 | 50-100 | >200 | >200 | 25-50 |
| 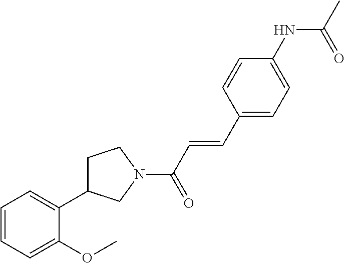 [SMILES: COc1ccccc1C1CCN(C(=O)/C=C/c2ccc(NC(C)=O)cc2)C1] | >100 | μM | >100 | μM | >200 | >200 | >200 | >200 | >200 |
| 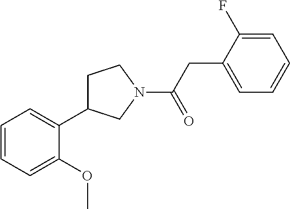 [SMILES: COc1ccccc1C1CCN(C(=O)Cc2ccccc2F)C1] | 4.6 | μM | 1.9 | μM | 50-100 | 50-100 | >200 | >200 | 50-100 |
| 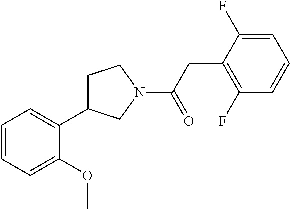 [SMILES: COc1ccccc1C1CCN(C(=O)Cc2c(F)cccc2F)C1] | 5.7 | μM | 1.6 | μM | 50-100 | 50-100 | >200 | >200 | 50-100 |
| 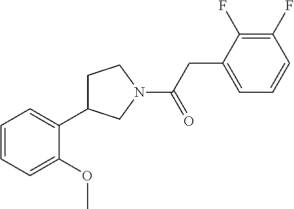 [SMILES: COc1ccccc1C1CCN(C(=O)Cc2cccc(F)c2F)C1] | 3 | μM | 7.4 | μM | 100 | 100 | >200 | >200 | 50 |
| 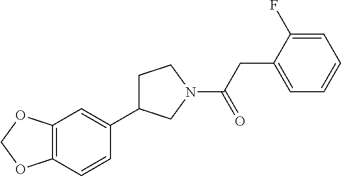 [SMILES: O=C(Cc1ccccc1F)N1CCC(c2ccc3c(c2)OCO3)C1] | 2.7 | μM | 32 | μM | >200 | >200 | >200 | >200 | 25-50 |
| 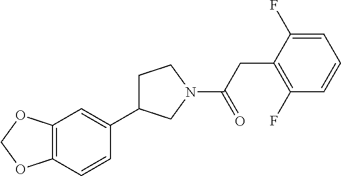 [SMILES: O=C(Cc1c(F)cccc1F)N1CCC(c2ccc3c(c2)OCO3)C1] | 2.3 | μM | 54 | μM | >200 | >200 | >200 | >200 | >200 |
| 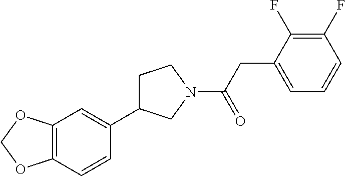 [SMILES: O=C(Cc1cccc(F)c1F)N1CCC(c2ccc3c(c2)OCO3)C1] | 5.5 | μM | 13.7 | μM | 100 | >200 | >200 | >200 | 25 |

It is to be understood that a wide range of changes and modifications to the embodiments described above will be apparent to those skilled in the art and are contemplated. It is, therefore, intended that the foregoing detailed description be regarded as illustrative rather than limiting, and that it be understood that it is the following claims, including all equivalents, that are intended to define the spirit and scope of the invention.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A compound according to Formula (I) 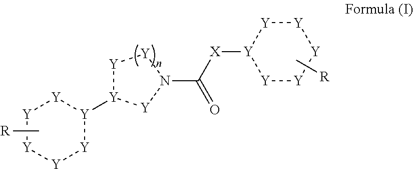
wherein:
each ---- independently represents a single bond or a double bond;
X=CH₂, CHR, CR₂, or CH═CH;
each Y is independently C, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2;
Formula (II) 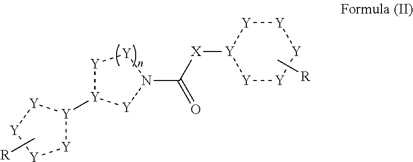
wherein:
each ---- independently represents a single bond or a double bond;
X=CH₂, CHR, CR₂, or CH═CH;
each Y is independently C, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2;
Formula (III) 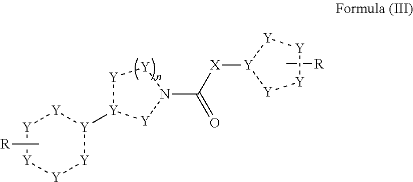
wherein:
each ---- independently represents a single bond or a double bond;
X=CH₂, CHR, CR₂, or CH═CH;
each Y is independently C, O, N, or S, in any order or combination;
each R^{a} and R^{b} is independently selected from H or null (if its corresponding Y is C and the C is sp² hybrodized), or taken together R^{a} and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2; or
Formula (IV): 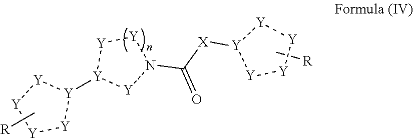
wherein:
each ---- independently represents a single bond or a double bond;
X=CH₂, CHR, CR₂, or CH═CH;
each Y is independently C, O, N, or S, in any order or combination;
each R^{a} and R^{b} is independently selected from H or null (if its corresponding Y is C and the C is sp² hybrodized), or taken together IV and R^{b} are ═O such that the corresponding CR^{a}R^{b} forms a carbonyl group; and
each R is independently H, halogen, alkyl, aryl, hydroxy, alkoxy, aroxy, amine, substituted amino, nitro, nitrile, amide, substituted amide, sulfonamide, substituted sulfonamide, carboxyl, substituted carboxyl;
n=0-2.

2. A compound of claim 1, wherein the 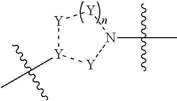
moiety of the compound is selected from the group consisting of:

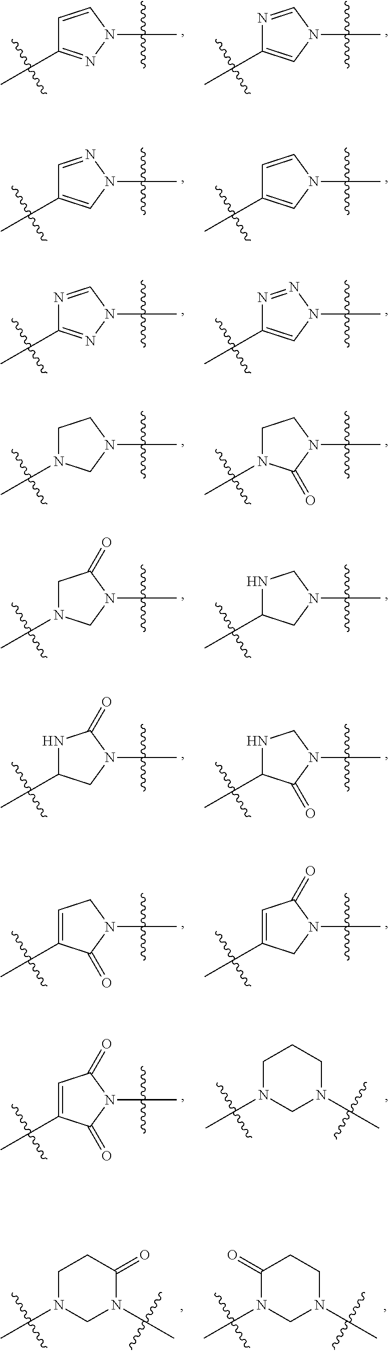

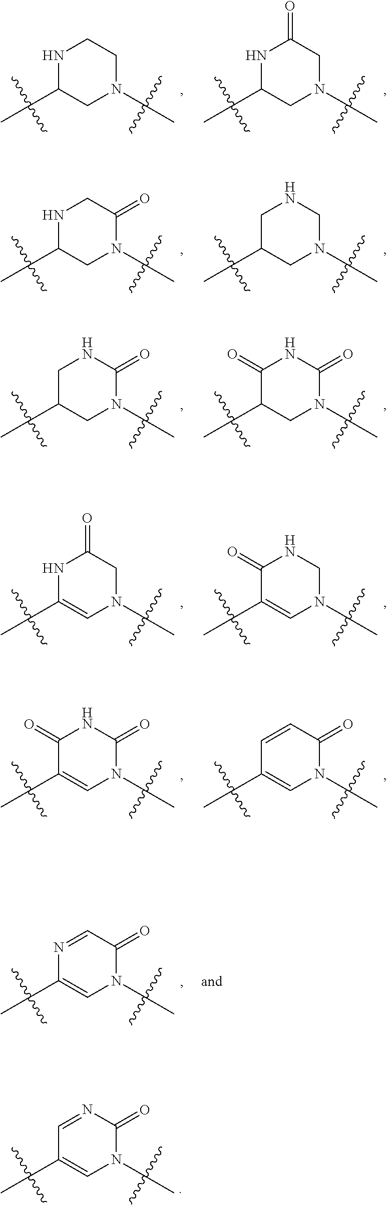

3. A compound of claim 1, wherein the compound is selected from the group consisting of: 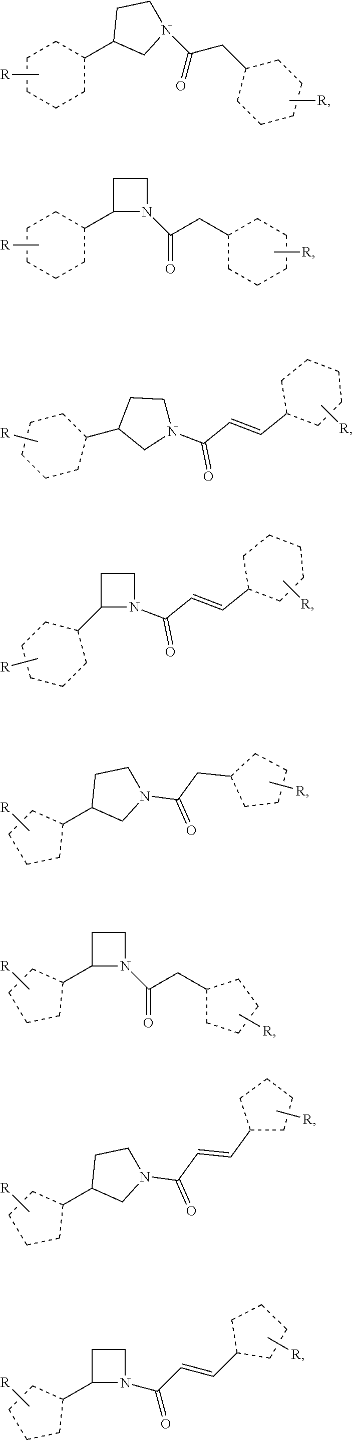 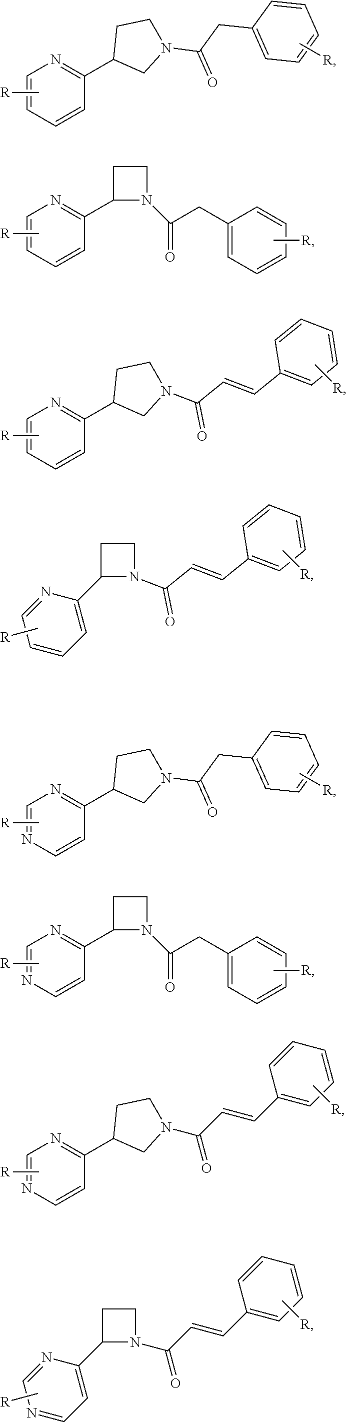 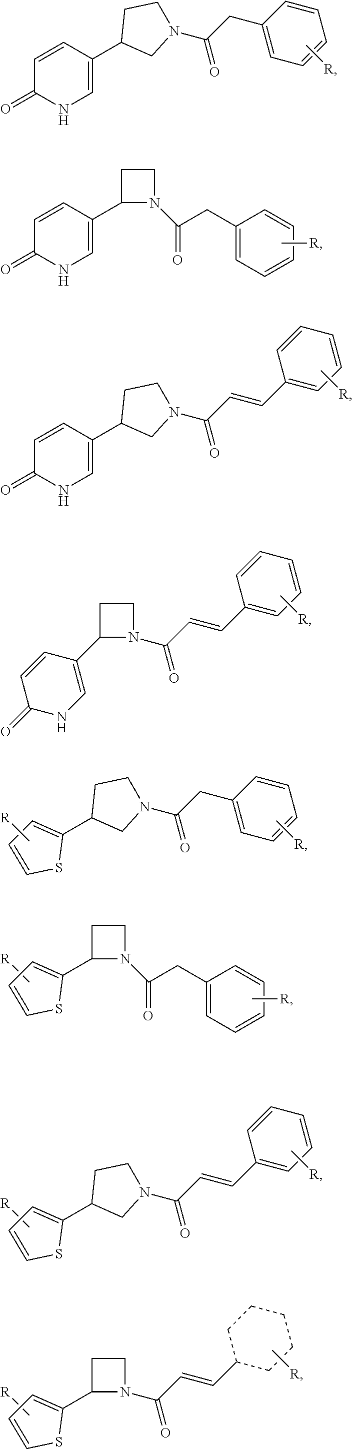 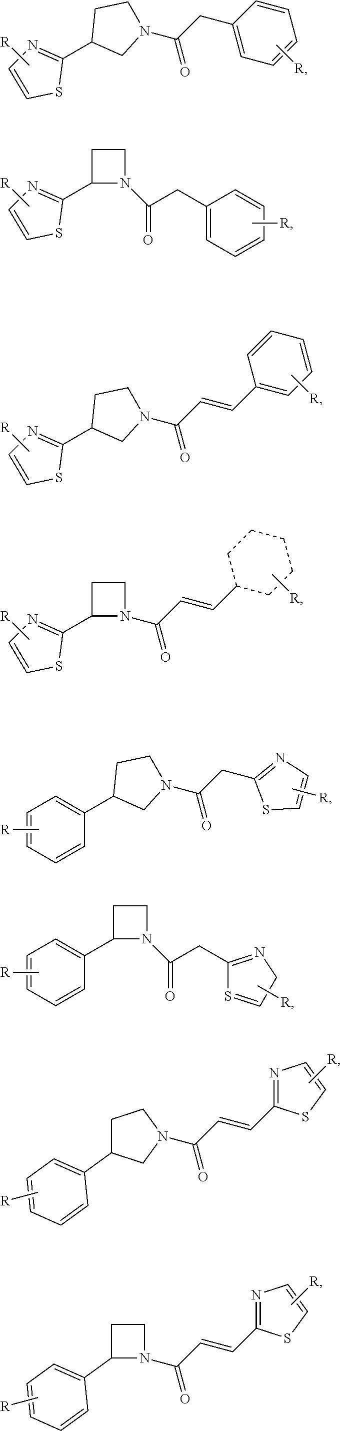 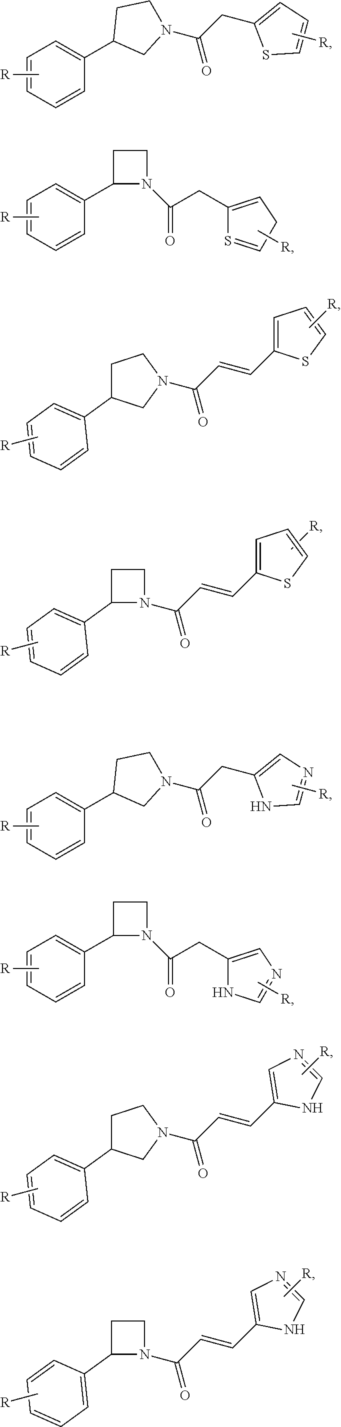 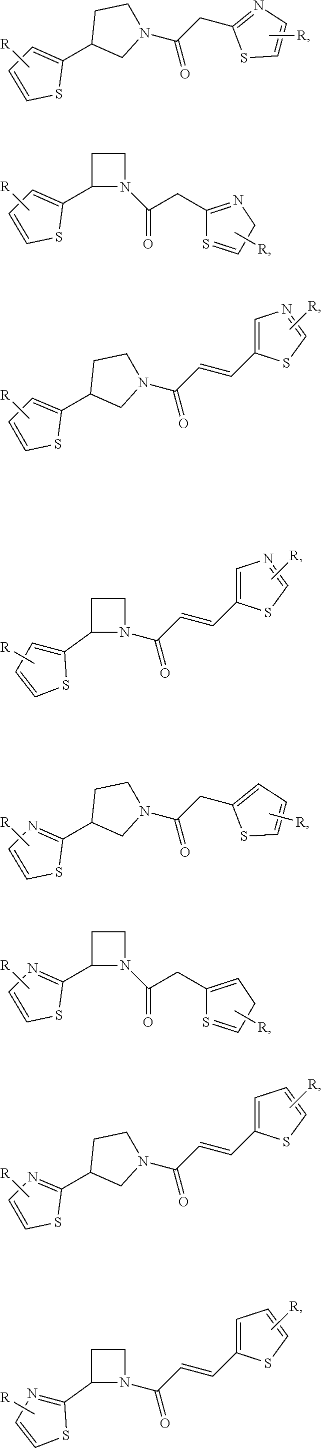 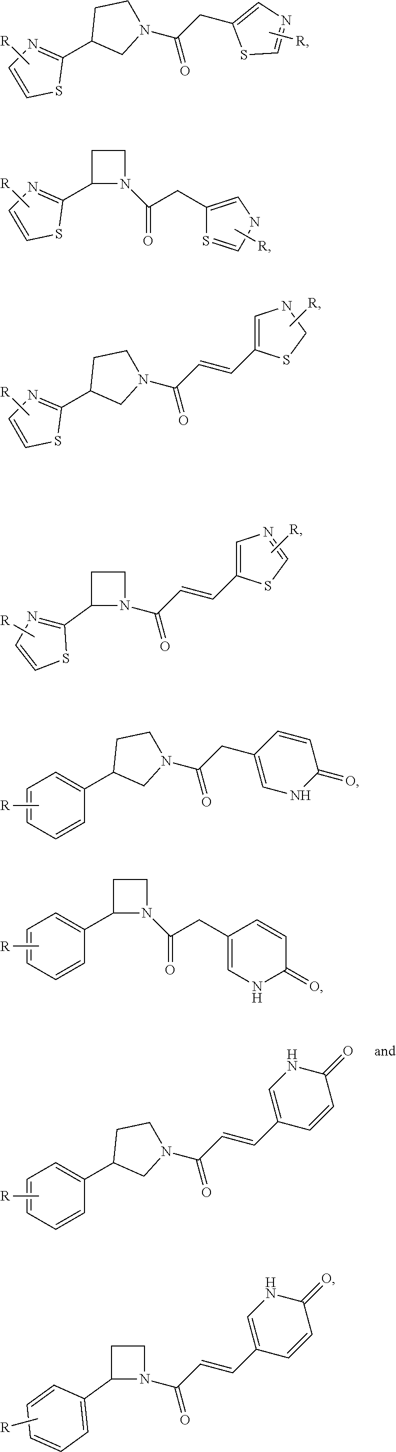
wherein
each ---- independently represents a single bond or a double bond; and
each R is independently H, halogen, alkyl, aryl, hydroxy, alkoxy, aroxy, amine, substituted amino, nitro, nitrile, or substituted amide.

4. A compound according to claim 1, wherein the compound has a structure selected from the group consisting of:

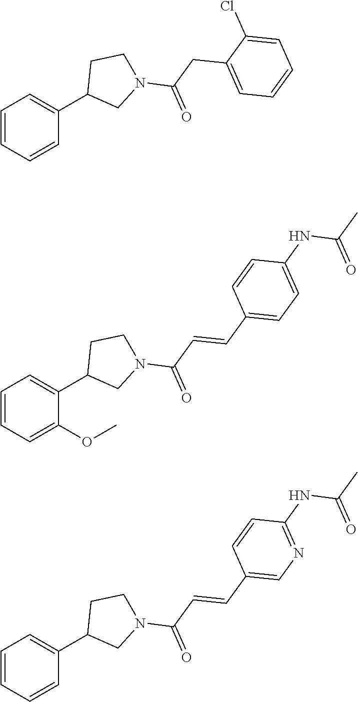 [SMILES: O=C(Cc1ccccc1Cl)N1CCC(c2ccccc2)C1 ; COc1ccccc1C1CCN(C(=O)/C=C/c2ccc(NC(C)=O)cc2)C1 ; CC(=O)Nc1ccc(/C=C/C(=O)N2CCC(c3ccccc3)C2)cn1]

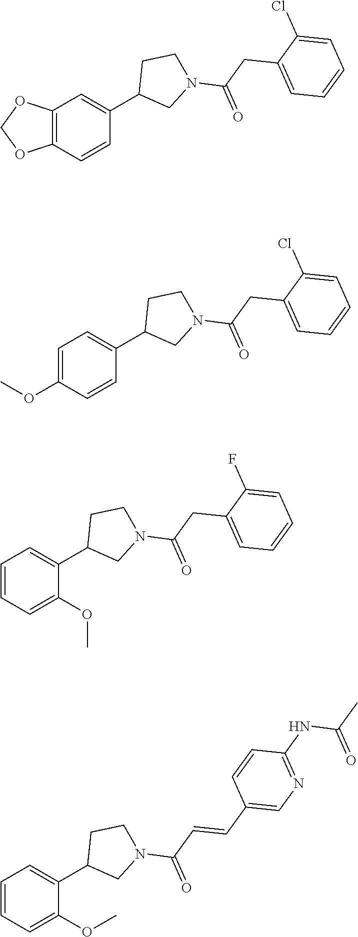 [SMILES: O=C(Cc1ccccc1Cl)N1CCC(c2ccc3c(c2)OCO3)C1 ; COc1ccc(C2CCN(C(=O)Cc3ccccc3Cl)C2)cc1 ; COc1ccccc1C1CCN(C(=O)Cc2ccccc2F)C1 ; COc1ccccc1C1CCN(C(=O)/C=C/c2ccc(NC(C)=O)nc2)C1]

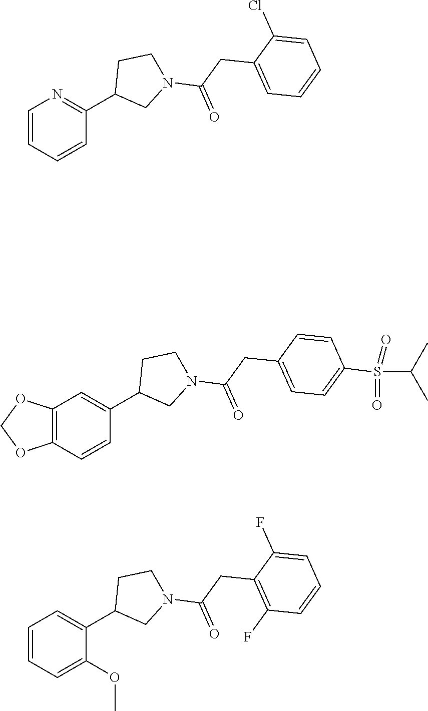 [SMILES: O=C(Cc1ccccc1Cl)N1CCC(c2ccccn2)C1 ; CC(C)S(=O)(=O)c1ccc(CC(=O)N2CCC(c3ccc4c(c3)OCO4)C2)cc1 ; COc1ccccc1C1CCN(C(=O)Cc2c(F)cccc2F)C1]

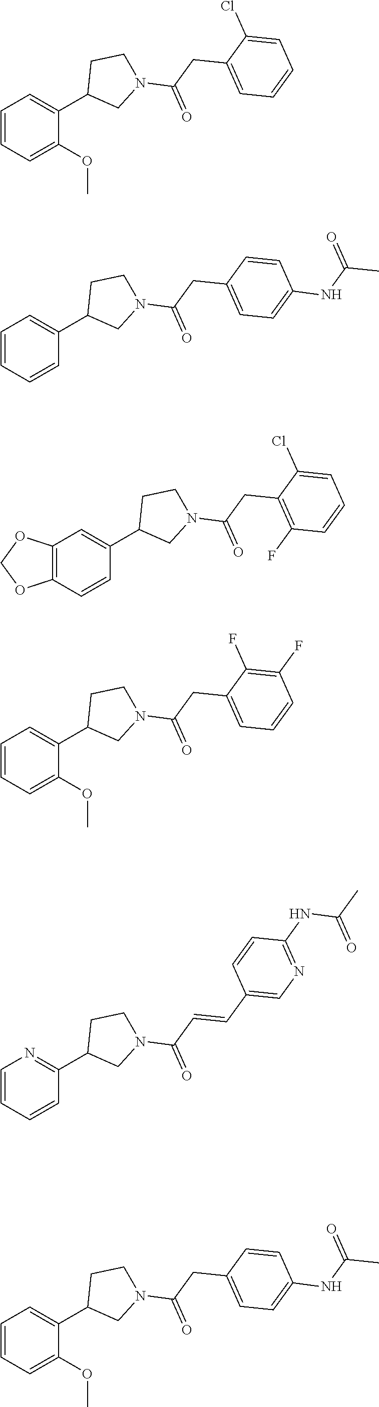 [SMILES: COc1ccccc1C1CCN(C(=O)Cc2ccccc2Cl)C1 ; CC(=O)Nc1ccc(CC(=O)N2CCC(c3ccccc3)C2)cc1 ; O=C(Cc1c(F)cccc1Cl)N1CCC(c2ccc3c(c2)OCO3)C1 ; COc1ccccc1C1CCN(C(=O)Cc2cccc(F)c2F)C1 ; CC(=O)Nc1ccc(/C=C/C(=O)N2CCC(c3ccccn3)C2)cn1 ; COc1ccccc1C1CCN(C(=O)Cc2ccc(NC(C)=O)cc2)C1]

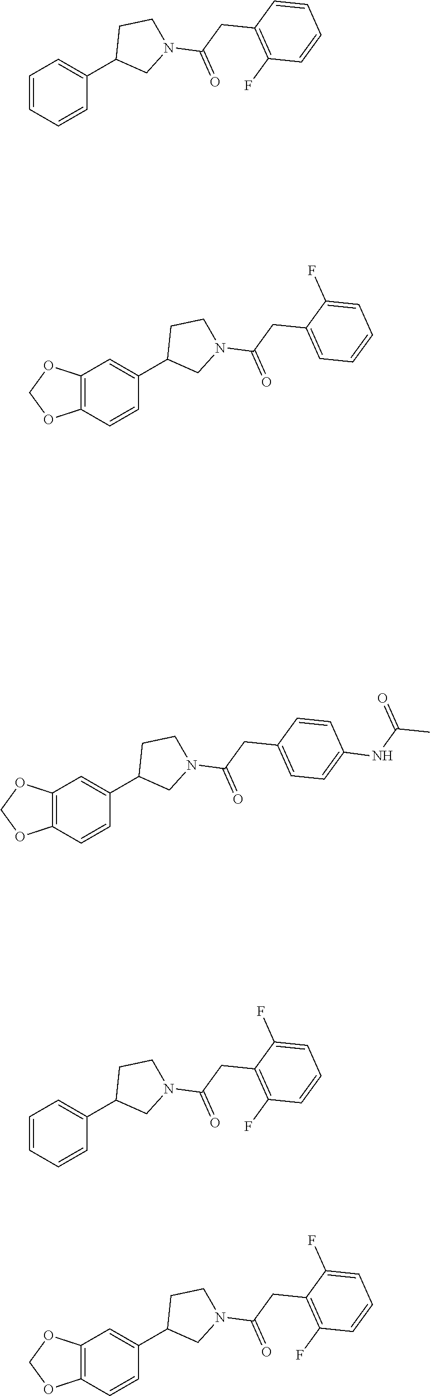 [SMILES: O=C(Cc1ccccc1F)N1CCC(c2ccccc2)C1 ; O=C(Cc1ccccc1F)N1CCC(c2ccc3c(c2)OCO3)C1 ; CC(=O)Nc1ccc(CC(=O)N2CCC(c3ccc4c(c3)OCO4)C2)cc1 ; O=C(Cc1c(F)cccc1F)N1CCC(c2ccccc2)C1 ; O=C(Cc1c(F)cccc1F)N1CCC(c2ccc3c(c2)OCO3)C1]

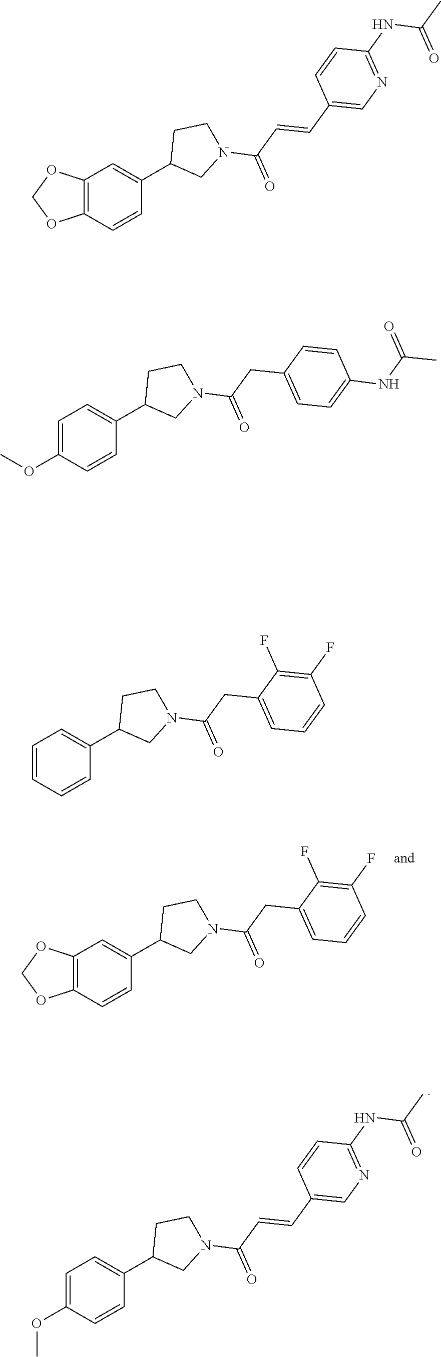

5. A compound according to claim 1, wherein the compound has a structure selected from the group consisting of:

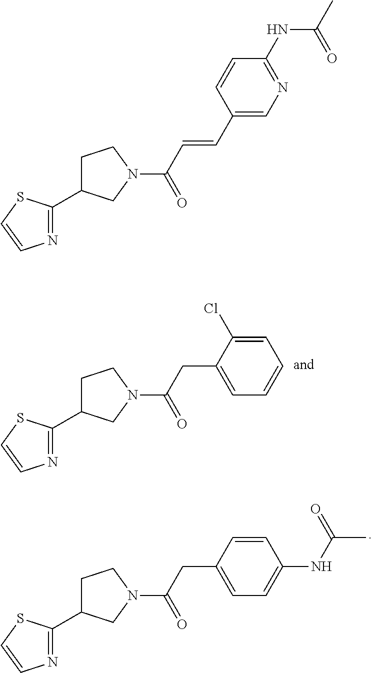 [SMILES: CC(=O)Nc1ccc(/C=C/C(=O)N2CCC(c3nccs3)C2)cn1 ; O=C(Cc1ccccc1Cl)N1CCC(c2nccs2)C1 ; CC(=O)Nc1ccc(CC(=O)N2CCC(c3nccs3)C2)cc1]

6. A tautomer, polymorph, stereoisomer, prodrug, solvate, pharmaceutically acceptable salt of a compound of claim 1.

7. A pharmaceutical composition comprising one or more compounds according to claim 1.

8. A method of treating or preventing a disease or disorder in a subject, the method comprising administering to the subject an effective amount of at least one compound of claim 1.

9. A method of treating or preventing a disease or disorder in a subject, the method comprising administering to the subject an effective amount of a pharmaceutical composition comprising a compound of claim 1.

10. The method of claim 9, wherein the disease or disorder is associated with an organism that utilizes FabI as its primary enoyl reductase.

11. The method of claim 10, wherein the organism is selected from the group consisting of: Francisella tularensis, Staphylococcus aureus, Bacillus anthraces, Plasmodium falciparum, Yersinia pestis, Enterococcus faecium, Staphylococcus epidermis, Staphylococcus saprophyticus, Clostridium perfringens, Bordetella pertussis, Brucella abortus, Brucella canis, Brucella melitensis, Brucella suis, Campylobacter jejuni, Haemophilus influenzae, Helicobacter pylori, Legionella pneumophila, Neisseria gonorrhoeae, Neisseria meningtidis, Rickettsiarickettsia, Salmonella enterica, Shigella sonnei, Vibrio cholera, Chlamydia trachomatis, Chlamydophila pneumonia, Chlamydophila psittaci, Mycobacterium tuberculosis, Mycobacterium leprae, Mycobacterium ulcerans, Acinetobacter baumannii, Chlamydophila pneumoniae, Escherichia coli, Klebsiella pneumoniae, Enterobacter, and Listeria monocytogenes.

12. The method of claim 8, wherein the subject is identified as having a disease or disorder associated with an organism that utilizes FabI as its primary enoyl reductase before the step of administering the compound.

13. The method of claim 9, wherein the subject is identified as having a disease or disorder associated with an organism that utilizes FabI as its primary enoyl reductase before the step of administering the pharmaceutical composition.

14. The method of claim 10, wherein the disease or disorder is selected from the group consisting of: a skin infection, an infection of a surgical wound, an infection of a trauma wound, a urinary tract infection, food poisoning, a gastrointestinal tract infection, an infection of an organ, pneumonia (a lung infection), osteomyelitis (a bone infection), endocarditis (a heart infection), phlebitis (an infection of veins and blood vessels), mastitis (an infection of breast and formation of abscesses), meningitis (a brain infection), an infection from and/or on an indwelling medical device (e.g., a joint prosthesis, a cardiovascular device, an artificial heart valve, etc.), a blood infection (e.g., a generalized life threatening blood infection), Toxic shock syndrome (TSS), bacteremia, septicemia, an intra-abdominal infection, a pelvic infection, a soft tissue infection, bacteremia, endocarditis, Anthrax, plague, malaria, a food borne illnesses, food poisoning, whooping cough, brucellosis, brucellosis in a canine, brucellosis in a livestock animal, brucellosis in a swine, camplyobacteriosis, an ulcer, a stomach infection, Legionnaires' disease, gonorrhea, meningococcal disease, rickettsial (spotted and/or typhus fevers) and related infections (e.g., anaplasmosis and ehrlichiosis), gastroenteritis, enteric fever, shigellosis, cholera, chlamydia, avian chlamydiosis, tuberculosis, leprosy, ulcerative skin disease, pharyngitis, bronchitis, coronary artery disease, atypical pneumonia, conjunctivitis, endocarditis, septic arthritis, CNS infections, ophthalmic infections, and listeriosis.
